SEQUENCE LISTING (6 sequences)

SEQ ID NO: 1 - DNA, 21 bp, Artificial Sequence
  Primer
gatcaaaaccacagcaggaac

SEQ ID NO: 2 - DNA, 18 bp, Artificial Sequence
  Primer
gccacgggaggaggcgag

SEQ ID NO: 3 - DNA, 18 bp, Artificial Sequence
  Probe
ccagctatttggtttgag

SEQ ID NO: 4 - DNA, 21 bp, Artificial Sequence
  Plasmid Construct
gcgccattctgacctctagag

SEQ ID NO: 5 - DNA, 21 bp, Artificial Sequence
  Plasmid Construct
gcgccattctagcctctagag

SEQ ID NO: 6 - DNA, 21 bp, Artificial Sequence
  Plasmid Construct
gcgccattctaacctctagag

(19) United States
(12) Patent Application Publication
(10) Pub. No.: US 2008/0207538 A1
(43) Pub. Date: Aug. 28, 2008

(54) Enhanced Production of Functional Proteins From Defective Genes

(76) Inventors: David S. Lawrence, Hartsdale, NY (US); Biao Xi, Bronx, NY (US)

Correspondence Address: AMSTER, ROTHSTEIN & EBENSTEIN LLP, 90 PARK AVENUE, NEW YORK, NY, 10016, US

(21) Appl. No.: 10/586,892

(22) PCT Filed: Mar. 7, 2005
(86) PCT No.: PCT/US2005/007365

§ 371 Date: Feb. 9, 2007

Related U.S. Application Data
(60) Provisional application No. 60/552,088, filed on Mar. 11, 2004.

(51) Int. Cl. A61K 31/7036; A61K 31/7028; A61P 35/00; A61P 43/00

(52) U.S. Cl. 514/41; 514/40; 514/230.2

(57) ABSTRACT

This invention provides methods for enhancing production in a subject of a functional protein from a gene disrupted by a mutation, for example by the presence of a premature stop codon or by an exon skipping mutation, comprising administering to the subject an amount of an agent effective to suppress the mutation and an amount of an agent effective to increase transcription of the gene.

### CROSS-REFERENCE TO RELATED APPLICATION

This application claims the benefit of U.S. Provisional Patent Application No. 60/552,088, filed Mar. 11, 2004, the content of which is hereby incorporated by reference into the subject application.

### STATEMENT OF GOVERNMENT SUPPORT

The invention disclosed herein was made with U.S. Government support under grant number R21 NS44429 from the National Institutes of Health, U.S. Department of Health and Human Services; Accordingly, the U.S. Government has certain rights in this invention.

### FIELD OF THE INVENTION

The present invention relates to the treatment of genetic disorders arising from mutations such as nonsense mutations, using the combination of an agent that suppresses the mutation and an agent that increases transcription.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

The termination of protein synthesis is signaled by the nucleic acid stop (nonsense) codons UAA, UAG, and UGA. Nonsense mutations occur when a sense codon is changed into one of the three stop codons. Nonsense mutations thus result in the premature termination of protein synthesis and the truncation or absence of a key protein product, and are associated with a host of genetic diseases (Barton-David et al., 1999; Clancy et al., 2001; Keeling et al., 2001; Sleat et al., 2001; Wilschanski et al., 2000). These diseases include thalassemia (α-globin and β-globin genes), hemophilia A and B (factor VIII and factor IX genes), von Willebrand's disease (vWF gene), p53 related cancers (p53 gene), colorectal cancers (APC, MSH1, and MSH2 genes), cystic fibrosis (CFTR gene), Duchenne muscular dystrophy (dystrophin gene), Tay-Sachs disease (hexosaminidase A gene), Wilms tumor (Wt1 gene), retinoblastoma (Rb gene), neurofibromatosis (NF1 and NF2 genes), ataxia telangiectasia (atmi gene), the lysosomal storage disease mucopolysaccharidosis I (IDUA gene), Hurler's syndrome, cystinosis, and late infantile neuronal ceroid lipofuscinosis.

Recently, gentamicin and other aminoglycoside antibiotics have been shown to suppress premature stop codon arrest by inducing the ribosome to read past the nonsense mutation via insertion of a random amino acid by a noncognate tRNA (Howard et al., 2000; Manuvaldova et al., 2000; Stephenson, 2001). Although the precise mechanism of nonsense mutation suppression remains to be established, gentamicin exerts its antibiotic action by targeting the 3OS ribosomal subunit, where it interferes with the initiation complex of protein formation (Yoshizawa et al., 1998). Presumably, a related binding event in mammalian cells enables gentamicin to actively promote premature stop codon suppression. Aminoglycosides have now been used to suppress nonsense mutations in human cell lines and animal models of Hurler's syndrome, Duchenne muscular dystrophy, late infantile neuronal ceroid lipofuscinosis, cystinosis, cystic fibrosis, mucopolysaccharidosis I, and p53 gene related disorders (Barton-David et al., 1999; Clancy et al., 2001; Helip-Wooley et al., 2002; Keeling et al., 2001, 2002; Sleat et al., 2001; Wilschanski et al., 2000). Gentamicin has also been used in patients with cystic fibrosis (Clancy et al., 2001; Wilschanski et al., 2000, 2003) and Duchenne muscular dystrophy (Politano et al., 2003).

Unfortunately, gentamicin therapy has limitations. First and foremost, suppression rates tend to be low and attempts to enhance these rates could have devastating genome-wide consequences by repressing bona fide stop codon signals. Second, ototoxicity and nephrotoxicity are known and serious side effects of gentamicin therapy. Finally, aminoglycoside antibiotics suppress stop codons with dramatically different efficiencies (UGA>UAG>UAA) and the ability to read past these codons is further dependent upon the local sequence context (Howard et al., 2000; Manuvakhova et al, 2000; Stephenson, 2001),

### SUMMARY OF THE INVENTION

The present invention is directed to a method for enhancing production in a subject of a functional protein from a gene disrupted by a mutation, for example by the presence of a premature stop codon in the coding region of the gene, comprising administering to the subject an amount of an agent effective to suppress the premature stop codon or the mutation, and an amount of an agent effective to increase transcription of the gene, so as to enhance production of the functional protein in the subject.

### BRIEF DESCRIPTION OF THE FIGURES

FIG. 1A-1C. Description of plasmid constructs. A: the atm/pA3LUC plasmid, B: the internal control plasmid containing the CMV promoter-driven renilla luciferase gene, and C: the three constructs containing stop codons inserted 43 bp from the start site of the coding region of firefly luciferase (1650 bp) (from top to bottom, respectively, SEQ ID NO:4-6). Opal stop codon—UGA; amber stop codon—UAG; ochre stop codon—UAA.

FIG. 2A-2B. Enhanced protein production from a defective gene following nonsense suppression and promoter activation. A: Gentamicin-induced suppression of a premature stop codon results in the formation of protein product (a hypothetical 25% suppression rate is shown in this example). B: Simultaneous treatment with gentamicin and a promoter activating drug can generate more protein product, even at the same suppression rate.

### DETAILED DESCRIPTION OF THE INVENTION

The subject invention is directed to a method for enhancing production in a subject of a functional protein from a gene disrupted by the presence of a premature stop codon in the coding region of the gene, comprising administering to the subject an amount of an agent effective to suppress the premature stop codon and an amount of an agent effective to increase transcription of the gene. As used herein, a “functional” protein means a protein that is able to carry out the same function as a protein normally produced from the gene in the absence of the disruption in the gene's coding region. As used herein, to “suppress” a mutation, for example the occurrence of a premature stop codon, encompasses both complete suppression and partial suppression of the premature stop codon or the mutation.

The agent that suppresses the premature stop codon can be an aminoglycoside antibiotic. Aminoglycoside antibiotics include, but are not limited to, gentamicin, geneticin, paromomycin, hygromycin, G-418, kanarnycin, amikacin and tobramycin. Preferred aminoglycoside antibiotics include gentamicin, geneticin, and amikacin. Gentamicin and amikacin have been reported to be more effective at suppressing nonsense mutations than tobramycin (Keeling and Bedwell, 2002). Sources for aminoglycoside antibiotics include: gentamicin, Schering-Plough, and Elkins-Sinn (Cherry Hill, N.J.); amikacin, Geneva Pharmaceuticals (Broornfield, Colo.); and tobramycin, Geneva Pharmaceuticals, and Eli Lilly and Company (Indianapolis, Ind.). In addition, assays for identifying other compounds that inhibit premature translation termination have been described (U.S. Pat. No. 6,458,538 B1). Another agent that can be used to suppress the premature stop codon is PTC124 (PTC Therapeutics Inc., South Plainfield, N.J.). PTC124 has been reported to be effective in animal models of cystic fibrosis and Duchenne muscular dystrophy, and has the advantage that it can be administered orally.

Gentamicin has previously been used to suppress premature stop codons in patients with cystic fibrosis, in the absence of administration of an agent effective to increase transcription of the gene, using different routes of administration (Clancy et al., 2001; Wilschanski et al., 2000, 2003). In one study (Clancy et al., 2001), gentamicin was administered intravenously at an initial dose of 2.5 mg/kg every 8 hours. Dosing was adjusted to achieve peak serum levels between 8-10 μg/ml and trough values <2 μg/ml. Treatment was continued for 7 days. In other patients, nasal administration was used (2 drops containing 0.3% gentamicin in each nostril, 3 times daily, for 14 day periods) (Wilschanski et al., 2000, 2003). Gentamicin has also been administered to a small number of patients with Duchenne muscular dystrophy caused by premature stop codon (Politano et al., 2003).

The agent that increases transcription of the gene can be an agent that activates a promoter of the gene. A “promoter” is a nucleic acid sequence that controls transcription of a nucleic acid. As used herein, to “activate” a promoter of a gene includes directly activating the promoter and/or indirectly activating the promoter, for example by decreasing suppression of the promoter, so that transcription of the gene is increased and more copies of messenger RNA (mRNA) are produced. The promoter can be a “constitutive” promoter that is active under most conditions, or an “inducible” promoter or a “tissue specific” promoter, which is active only under certain specific or regulated conditions. In one embodiment, the agent that activates the promoter of the gene is a fluorinated quinolone. The fluorinated quinolone can be ofloxacin. In another embodiment, the agent that activates the promoter of the gene is thioguanine. Thioguanine and ofloxacin can both be obtained from Sigma (St. Louis, Mo.). Thioguanine is also available from GlaxoSmithKline (Research Triangle Park, N.C.), and ofloxacin is also available from ORTHO-McNEIL Pharmaceutical (Raritan, N.J.).

An activator of a promoter of a gene can be readily identified by one skilled in the art using methods similar to those described herein in Experimental Details. For example, the promoter can be cloned, attached to luciferase cDNA, and transfected into an appropriate cell line, which can then be used to simultaneously screen multiple candidate activators of the promoter. A library of nearly 400 drugs approved by the Federal Drug Administration (FDA) that can be used as candidate agents can be found in the Supporting Information to Xi et al. (2004).

Preferably, the production of functional protein is enhanced by a factor of at least 7-fold relative to an untreated control. More preferably, the production of functional protein is enhanced by a factor of at least 10-fold or 20-fold or 30-fold relative to an untreated control.

Preferably, the production of functional protein is enhanced by a factor of at least 2-fold relative to the production obtained using only the agent that suppresses the premature stop codon. More preferably, the production of functional protein is enhanced by a factor of at least 3-fold relative to the production obtained using only the agent that suppresses the premature stop codon.

Preferably, the production of functional protein is enhanced to a level that corresponds to at least 10% of the level of functional protein generated from a corresponding native gene in which a premature stop codon is absent.

Preferably, the agent that suppresses the premature stop codon is administered at a dose lower than the dose that would be required to produce the same amount of functional protein in the absence of the agent that increases transcription. Preferably, the lower dose of the agent that suppresses the premature stop codon results in decreased toxicity.

The disruption of the gene can be associated with a genetic disorder, including, but not limited to, thalassemia, hemophilia A, hemophilia B, von Willebrand's disease, a p53 related cancer or disorder, a colorectal cancer, cystinosis, cystic fibrosis, Duchenne muscular dystrophy, Tay-Sachs disease, Wilms tumor, retinoblastoma, neurofibromatosis, ataxia telangiectasia, Hurler's syndrome, mucopolysaccharidosis I, and late infantile neuronal ceroid lipofuscinosis.

In one embodiment, the genetic disorder is ataxia telangiectasia, and the agent that activates a promoter of the gene is a fluorinated quinolone, such as ofloxacin, or thioguanine. The agent that suppresses the premature stop codon can be, but is not limited to, gentamicin or geneticin.

Preferably, the genetic disorder is treated by a method disclosed herein. As used herein, to “treat” a genetic disorder means to reduce or eliminate a symptom and/or cause of the genetic disorder in a subject.

The nonsense mutation can occur in a tumor suppressor gene. Such genes include, but are not limited to, BRCA1, BRCA2, PTEN, NF1, NF2, MLH1, MLH2, VHL, WT1, TSC1, TSC2, and ATM. Preferably, the enhanced production of the functional protein that results from practicing a method of the present invention is effective to treat a tumor in the subject. As used herein, to “treat” a tumor in a subject means to keep the tumor from growing, to reduce the size of the tumor, or to eliminate the tumor in the subject.

The techniques of the present invention can also be combined with techniques that inhibit the decay of RNA (e.g., U.S. Pat. No. 6,458,538 B1).

The present invention also encompasses methods for enhancing production in a subject of a functional protein, where production of the protein is disrupted by a genetic mutation, comprising administering to the subject an amount of an agent effective to suppress the genetic mutation and/or correct a defect caused by the mutation, and an amount of an agent effective to increase transcription of the gene. Such genetic mutations include missense, frame shift, and exon skipping mutations, which disrupt production of the protein from the gene. A missense mutation results in a protein in which one amino acid is substituted for another. Frameshift mutations result from the addition or deletion of nucleotides that are not a multiple of three, leading to a change in the coding region of the gene and typically the introduction into the protein of a sequence of amino acids that is unrelated to the sequence normally occurring in the protein. An exon skipping mutation occurs when amino acids encoded by an exon are not incorporated into the protein. The agent that increases transcription of the gene can be an agent that activates a promoter of the gene. The genetic mutation can be associated with a genetic disorder. Preferably, the combined therapy approach of the present invention is effective to treat the genetic disorder.

Agents that suppress exon skipping mutations and/or correct the defect caused by the mutation (Buratti et al., 2003; Khoo et al., 2003) include a synthetic hybrid peptide-nucleic acid molecule (Cartegni and Krainer, 2003), a 2′-O-methyl phosphorothioate oligonucleotide combining an antisense sequence and an exonic splicing enhancer sequence (Skordis et al., 2003), sodium butyrate (Chang et al., 2001), and the anthracycline aclarubicin (Andreassi et al., 2001). The hybrid peptide-nucleic acid molecule can include both an antisense moiety and a arginine-serine (RS) domain. Sodium butyrate has been used with minimal toxicity to treat patients with sickle-cell anemia (Perrine et al., 1993; reviewed in Chang et al., 2001). Exon skipping mutations (Cartegini et al., 2002) which occur in the Survival Motor Neuron gene SMN2 are associated with spinal muscular atrophy (SMA). Mutations in SMN1 cause SMA, while SMN2 is believed to modify its severity (reviewed in Andreassi et al., 2001 and Skordis et al., 2003).

The subject can be a mammal. In different embodiments, the mammal is a mouse, a rat, a cat, a dog, a horse, a sheep, a cow, a steer, a bull, livestock, a primate, a monkey, or preferably a human.

This invention will be better understood from the Experimental Details which follow. However, one skilled in the art will readily appreciate that the specific methods and results discussed are merely illustrative of the invention as described more fully in the claims which follow thereafter.

### Experimental Details

### Introduction

Initial efforts focused on ataxia telangiectasia (A-T), a rare childhood disorder characterized by the eventual loss of motor control, moderate to severe inmunodeficiency, premature aging, and a pronounced predisposition to cancer (Rotman and Shiloh, 1998). Like many rare childhood diseases, it is the absence, rather than the overabundance, of a key protein that is responsible for these devastating symptoms. Shiloh and his colleagues identified the gene (atm) that is defective in A-T (Rotman and Shiloh, 1998). A variety of atm deletion, nonsense, and missense mutations have been subsequently reported, the vast majority (>70%) of which result in protein truncation (Concannon and Gatti, 1997). Truncated ATM protein is unstable and therefore not detected in most A-T cell lines.

### Materials, Methods and Procedures

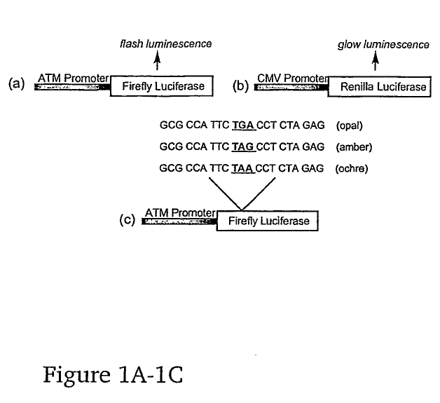

Plasmid construction. The wild type human fibroblast cell line GM02037 (Coriell Cell Repositories, NJ) was used as the DNA template for atm promoter amplification. In brief, the Qiagen Blood & Cell Culture DNA Mini kit was used to purify genomic DNA. ATM gene promoter primers, which were used to amplify the desired fragment DNA, were designed based on the sequence in Genebank. The forward primer sequence employed was GATCAAAACCACAGCAGGAAC (SEQ ID NO:1) and the reverse primer was GCCACGGGAGGAGGCGAG (SEQ ID NO:2). PCR was carried out using the Roche Expand High Fidelity PCR system. The amplified atm promoter region was then cloned into the TOPO2.1 PCR vector (Invitrogen) for sequencing verification and subsequently subcloned into the promoterless plasmid PA3Luc. Firefly luciferase cDNA was mutated (QuikChange Site-Directed Mutagenesis Kit) in the desired region to furnish the three stop codons, as shown as FIG. 1(C). The CMV driven Renilla luciferase plasmid was purchased from Promega as the internal control for the dual luciferase assay.

Cell culture and transfection. Human Kidney HEK293T cell was a gift from Professor Philipp Scherer in the Department of Cell Biology at the Albert Einstein College of Medicine. All other cell lines were purchased from Coriell Cell Repositories, NJ. HEK293T cells were maintained in Dulbecco's Modified Eagle Medium with 1 g/L D-glucose and 10% fetal bovine serum. The Human AT fibroblast cell line GM05823 was cultured in MEM medium with 1 g/L D-glucose and 15% fetal bovine serum. The Human lympoblastoid cell lines GM 13810 and GM13860 were cultured in RPMI 1640 medium with 10% heat inactivated fetal bovine serum and 1 g/L of D-glucose. All cell lines were incubated at 37° C. in a humidified 5% CO2 atmosphere.

Transfections were performed using GeneJammer reagent (Stratagene) based on the protocol from the manufacturer. In brief, approximately 15×105 HEK293T cells were seeded into white 96 well plates (Corning) and grown for less than 24 hours. Cells were transfected with 0.6 μL of GeneJammer and 0.1 μg plasmids. In the dual luciferase assay, the plasmid ratio of ATM promoter-driven firefly luciferase to CMV promoter-driven Renilla luciferase was 30:1. In the dual luciferase experiments that utilized the stop codon-containing firefly luciferase genes, the ratio was 40:1. On the second day after transfection, the medium in each well was changed, and the promoter and/or nonsense-suppressing agents added to the wells for a 24 hour incubation period. The cells were subsequently lysed and promoter activity/nonsense suppression efficiency then assessed via the dual luciferase assay.

Dual luciferase assay. The dual luciferase kit was purchased from Promega and the protocol employed was conducted according to the directions supplied by the manufacturer. In brief, cells were washed with PBS and lysates were prepared using passive lysis buffer. Luminescence was determined with a Molecular Devices Lmax 96 well plate luminometer. Light emission was measured after each of the 100 μL luminescence substrates and/or stop solutions were injected. ATM promoter activity was calculated by the ratio of relative light units of flash (firefly) to glow (renilla) luminescence in each well, which was compared with control wells (cultured only in medium with plasmid but no drug treatment) in each plate. In the case of the stop codon suppression studies, controls also included cells containing the plasmids without the inserted stop codon, which were treated with nonsense suppressing and/or promoter activating agents. All individual treatments in a given experiment were performed in triplicate and all experiments repeated 3-10 times.

Drug library and treatment. Drugs were purchased from a variety of commercial sources and dissolved in appropriate solvents (DMSO, water, dioxane, or methanol) to furnish 10 mM stock solutions (8×12 formatted stored in deep well plates). Diluted stocks (1 mM and 100 μM in 0.1% BSA, 1 mM Tris, pH 7.5 sterilized buffer) of the starting 10 mM library were prepared using a Packard Multiprobe II HT EX liquid handling robot. These stock solutions were subsequently transferred into a series of 8×12 formatted shallow well plates to avoid exposure to repetitive freeze-thaw cycles. All plates were kept −20° C. The drug solutions were thawed at room temperature and used at the final concentrations of 2.5 μM, 25 μM, and 250 μM. Before the drugs were employed to treat cells, the solutions in the individual wells were agitated using a multichannel pipettor. The drug solutions were subsequently added to cells cultured in wells of 96 well plates (where the final DMSO concentration is less than 2.5%).

mRNA Quantitation. Single-stranded cDNAs were synthesized by reverse transcription, using a First Strand cDNA Synthesis kit (Invitrogen), and then used as a template in polymerase chain reactions. ATM mRNA was primarily quantified using real time polymerase chain reaction (PCR). In brief, cells were treated with the desired drugs or combinations thereof under conditions identical to those described above for the dual luciferase assay. Cells were lysed and RNA extracted with the TRIzol reagent (Invitrogen). Approximately 1 μg of total RNA in each sample was subjected to CDNA synthesis using the First Strand cDNA Synthesis kit (Invitrogen). In the real time PCR reaction, the FAM labeled MGB probe, CCAGCTATTTGGTTTGAG (SEQ ID NO:3), was designed by the Primer Express software from Applied Biosystems (AB) and synthesized by AB. The human β-actin MGB probes, VIC or FAM fluorescently labeled, were purchased from AB. All other reaction reagents were purchased from AB. PCR reactions were performed using the ABI PRISM 7000 Sequence Detection System in triplicate. Some samples were also tested on the ABI 7700 Sequence Detection System for verification and essentially identical results were obtained with both real time PCR instruments. All collected data were compared with a 3-actin control. In addition to real time PCR, the branched chain DNA (bDNA) RNA quantitation method was employed as well. Briefly, the QuantiGene kit as well as the customer designed atm probe sets were purchased from Genospectra. The latter probes target the same region on the atm message RNA region as the real time PCR probes. The cells were treated as described above for the dual luciferase assay. Cell lysates were transferred into a capture plate and atm mRNA allowed to hybridize with the probe set at 53° C. overnight. After 16-20 hours, the signal was amplified and subsequently read using a Molecular Devices Lmax luminometer. The internal human β-actin probe sets were purchased from Genospectra. All experiments were performed in triplicate.

### Results and Discussion

A cell-based screen for atm promoter enhancing agents was developed by inserting the 700 bp atm promoter into pA3LUC, a promoterless plasmid containing a firefly luciferase cDNA reporter (LUC) (FIG. 1A-1C). The human embryonic kidney 293T cell line was transiently co-transfected with the atm/pA3LUC plasmid and a plasmid containing the gene encoding renilla luciferase driven by the constitutive CMV promoter (a commonly employed internal control). The transfected cells were then plated into individual wells of 96 well plates. A library of nearly 400 FDA approved drugs was assembled and individually screened these for their ability to activate the atm promoter. The obvious advantage associated with FDA-approved drugs is their established safety profile, which markedly reduces the expenses and lead-time associated with their application to disorders other than those for which they were originally intended. In addition, this structurally diverse family of chemicals is among the best understood of all biologically active compounds. Consequently, if a specific member of the FDA-approved family of drugs activates the expression of a specific gene, then there is a good likelihood that its mechanism of action can be deduced from the existing scientific literature.

Wells containing the transiently transfected 293T cells were treated with individual members at varied concentrations (0.25, 2.5, 25, and 250 μM) of the library of FDA-approved drugs. In addition, a series of controls was performed in each plate, including untreated (i.e. 5 mM glucose) and 25 mM glucose-exposed 293T cells. High glucose levels up-regulate ATM message and protein levels. The amount of renilla luciferase activity is indicative of the number of cells present in a given well, whereas the quantity of firefly luciferase activity represents the influence of the various drugs on atm promoter activity. In addition to 25 nm glucose, several FDA approved drugs activate the atm promoter, including the fluorinated quinolone ofloxacin. Fluorinated quinolones are known to target eukaryotic topoismerase II, an enzyme that catalyzes the interpenetration of DNA strands by introducing transient double strand breaks. Consequently, one possible mechanism by which ofloxacin activates the atm promoter is via the induction of double strand breaks, which is consistent with the role of ATM in DNA double strand break recognition and/or repair (Hooper, 2001; Rowe et al., 2001; Shiloh, 2003). If this mechanism is correct then the production of ATM protein would need to be sufficient to offset any drug-induced double strand breaks. However, several other drugs induce atm promoter activity, including the antimetabolite thioguanine (Bokkerink et al., 1993).

The effect of ofloxacin on native atm message levels was subsequently examined in 293T cells as well as in lymphoblastoid and primary fibroblast cell lines derived from A-T patients (Table 1). Message levels are enhanced 2.4±0.1 fold in 293T cells treated with ofloxicin for 24 hours. By contrast, an up to nearly 6-fold increase is observed in ATM-deficient (A-T) cell lines. The latter results offer the intriguing possibility that atm message levels are elevated more dramatically in an ATM-deficient environment. These results validate the use of promoter-activating agents identified in the luciferase screen to upregulate the intracellular levels of the corresponding atm message.

An evaluation was made of the ability of aminoglycosides, in combination with promoter activating agents, to augment read-through of a stop codon inserted into the reading frame of firefly luciferase (FIG. 1C). Both gentamicin, and its structurally related congener geneticin, enhance stop codon read-through by 6- to 15-fold relative to untreated controls (Table 2). By contrast, the combined use of a nonsense-suppressing agent (i.e. gentamicin or geneticin) with a promoter-activating drug (i.e. ofloxacin or thioguanine) furnishes read-through levels that can reach 30-fold greater than that observed with untreated cells. The latter, to a rough approximation, appears to reflect the enhanced message levels produced by the promoter-activating agent in 293T cells. In addition, the promoter-activation strategy provides enhanced read-through for all three stop codons, suggesting that the strategy outlined herein should prove applicable to nonsense mutations in general. Finally, in the most efficient case (gentamicin/thioguanine/ofloxacin; UGA stop codon), the level of functional luciferase induced from a defective gene is 10% of that generated from the corresponding native gene (i.e. no nonsense mutation present). This level of expression may prove noteworthy since it constitutes a significant fraction of the 50% level present in the vast majority of carriers of genetic disorders (i.e. one defective allele), individuals who generally do not display a disease phenotype.

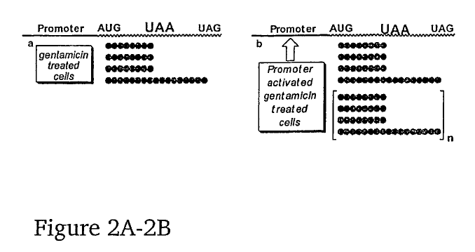

The strategy disclosed herein can augment the efficacy of aminoglycoside-induced nonsense suppression by enhancing target protein formation and by reducing the amount of aminoglycoside required for activity. FIG. 2A illustrates the gentamicin-induced suppression of a stop codon and the consequent synthesis of a protein with a hypothetical 25% suppression rate. Larger amounts of protein can be produced, even at the same nonsense suppression rate, if suppression is conducted in the presence of a promoter-activating agent (FIG. 2B). The latter can enhance the quantity of message available for translation and thereby generate more target protein.

In summary, a promoter-activating agent, in combination with an aminoglycoside antibiotic, can stimulate enhanced production of functional protein from nonsense codon-containing genes. A library of FDA-approved drugs was screened to identify agents that activate the atm promoter. FDA approved drugs enjoy the obvious advantage that they have already been accepted for human use and thus can be rapidly applied to treat additional diseases or ailments. In addition, the biochemical mechanism of action of the overwhelming majority of these agents is extremely well understood. Consequently, the global response of a given promoter to a broad-spectrum of FDA-approved drugs may help to delineate the signaling pathways that influence the activity of that promoter.

TABLE 1
Fold-Increase in atm Message Level as a Function
of Time Following Treatment with Ofloxacin.

| | Fold-increase | | |
|---|---|---|---|
| | (atm message levels)^{a} | | |
| Cell Lines | 2 hours | 6 hours | 24 hours |
| GM13810 (A-T)^{b} | 1.2 ± 0.2 | 2.3 ± 0.3 | 5.4 ± 0.7 |
| GM13860 (A-T)^{b} | 3.8 ± 0.9 | 2.3 ± 0.3 | 3.3 ± 0.3 |
| GM05823 (A-T)^{c} | 2.2 ± 0.1 | 3.0 ± 0.2 | 5.8 ± 0.4 |

^{a}As measured by RT-PCR, which were performed twice, each time in triplicate. Fold-increase relative to the corresponding untreated cell line.
^{b}Lymphoblastoid cell lines.
^{c}Fibroblast cell line.

TABLE 2
Fold-Increase in Stop Codon Read-through in 293T Cells
Treated with Aminoglycoside Alone or Aminoglycoside
in Combination with Promoter Activating Agent.

Fold-increase (read-through)^{a}

| Protocol | opal | amber | ochre |
|---|---|---|---|
| Geneticin | 6.5 ± 0.8 | 14.9 ± 1.9 | 7.9 ± 3.0 |
| Gentamicin | 8.7 ± 1.2 | 8.2 ± 1.8 | 6.7 ± 1.5 |
| Ofloxacin/Gentamicin | 14.3 ± 1.2 | 17.5 ± 1.9 | 9.6 ± 2.1 |
| Thioguanine/Gentamicin | 32.1 ± 6.2 | 29.2 ± 5.9 | 18.3 ± 2.3 |
| Ofloxacin/Geneticin | 14.6 ± 3.5 | 21.1 ± 1.1 | 7.1 ± 0.4 |

^{a}As measured by the dual luciferase assay (in triplicate) and relative to untreated 293T cells transfected with the indicated stop codon-containing gene.

### REFERENCES

Andreassi C, Jarecki J, Zhou J, Coovert D D, Monani U R, Chen X, Whitney M, Pollok B, Zhang M, Androphy E, Burghes A H. Aclarubicin treatment restores SMN levels to cells derived from type I spinal muscular atrophy patients. Hum Mol Genet. 2001 Nov 15; 10(24):2841-9.

Barton-Davis, E. R., Cordier, L., Shoturma, D. I., Leland, S. E., Sweeney, H. L. Aminoglycoside antibiotics restore dystrophin function to skeletal muscles of mdx mice. J. Clin. Invest. 104: 375-381, 1999.

Bokkerink:, J. P., Stet, E. H., De Abreu, R. A., Damen, F. J., Hulscher, T. W., Bakker, M. A., van Baal, J. A. 6-Mercaptopurine: cytotoxicity and biochemical pharmacology in human malignant T-lymphoblasts. Biochem. Pharmacol. 45:1455-1463, 1993.

Buratti E, Baralle F E, Pagani F. Can a ‘patch’ in a skipped exon make the pre-mRNA splicing machine run better? Trends Mol Med. 2003 Jun; 9(6):229-32.

Cartegni L, Chew S L, Krainer A R. Listening to silence and understanding nonsense: exonic mutations that affect splicing. Nat Rev Genet. 2002 Apr; 3(4):285-98.

Cartegni L, Krainer A R. Correction of disease-associated exon skipping by synthetic exon-specific activators. Nat Struct Biol. 2003 Feb; 10(2):120-5.

Chang J G, Hsieh-Li H M, Jong Y J, Wang N M, Tsai C H, Li H. Treatment of spinal muscular atrophy by sodium butyrate. Proc Natl Acad Sci U S A. 2001 Aug 14; 98(17):9808-13.

Clancy, J. P., Bebok, Z., Ruiz, F., King, C., Jones, J., Walker, L., Greer, H., Hong, J., Wing, L., Macaluso, M., Lyrene, R., Sorscher, E. J., Bedwell, D. M. Evidence that systemic gentamicin suppresses premature stop mutations in patients with cystic fibrosis. Am. J. Respir. Crit. Care Med. 163: 1683-1692, 2001.

Concannon, P. and Gatti, R. Diversity of ATM gene mutations detected in patients with ataxia-telangiectasia. Hum. Mutat. 10:100-107, 1997.

Helip-Wooley A, Park M A, Lemons R M, Thoene J G. Expression of CTNS alleles: subcellular localization and aminoglycoside correction in vitro. Mol. Genet. Metab. 75(2):128-33, 2002.

Hooper, D. C. Mechanisms of action of antimicrobials: focus on fluoroquinolones, Clin. Infect. Diseases 32 (Suppl 1): S9-S15, 2001.

Howard, M. T., Shirts, B. H., Petros, L. M., Flanigan, K. M., Gesteland, R. F., Atkins, J. F. Sequence specificity of aminoglycoside-induced stop condon readthrough: potential implications for treatment of Duchenne muscular dystrophy. Ann. Neurol. 48: 164-169, 2000.

Keeling K M. and Bedwell D M. Clinically relevant aminoglycosides can suppress disease-associated premature stop mutations in the IDUA and P53 cDNAs in a mammalian translation system. J Mol Med. 2002 Jun; 80(6):367-76. Epub 2002 Jan 25.

Keeling, K. M., Brooks, D. A., Hopwood, J. J., Li, P., Thompson, J. N., Bedwell, D. M. Gentamicin-mediated suppression of Hurler syndrome stop mutations restores a low level of alpha-L-iduronidase activity and reduces lysosomal glycosaminoglycan accumulation. Human Mol. Gen. 10: 291-299, 2001.

Khoo B, Akker S A, Chew S L. Putting some spine into alternative splicing. Trends Biotechnol. 2003 Aug; 21(8):328-30.

Manuvakhova, M., Keeling, K., Bedwell, D. M. Aminoglycoside antibiotics mediate context-dependent suppression of termination codons in a mammalian translation system. RNA 6: 1044-1055, 2000.

Perrine S P, Ginder G D, Faller D V, Dover G H, Ikuta T, Witkowska H E, Cai S P, Vichinsky E P, Olivieli N F. A short-term trial of butyrate to stimulate fetal-globin-gene expression in the beta-globin disorders. N Engl J Med. 1993 Jan 14; 328(2):81-6.

Politano L, Nigro G, Nigro V, Piluso G, Papparella S, Paciello O, Comi L I. Gentamicin adiministration in Duchenne patients with premature stop codon. Preliminary results. Acta Myol. 2003 May; 22(1):15-21.

Rotman, G. and Shiloh, Y. ATM: from gene to function. Hum. Mol. Genet. 7: 1555-1563, 1998.

Rowe, T. C., Weissig, V., Lawrence J. W. Mitochondrial DNA metabolism targeting drugs. Advan. Drug Deliv. Rev. 49: 175-187, 2001.

Shiloh, Y. ATM and related protein kinases: safeguarding genome integrity. Nat. Rev. Cancer 3: 155-168, 2003.

Skordis L A, Dunckley M G, Yue B, Eperon I C, Muntoni F. Bifunctional antisense oligonucleotides provide a trans-acting splicing enhancer that stimulates SMN2 gene expression in patient fibroblasts. Proc Natl Acad Sci U S A. 2003 Apr 1; (7):4114-9. Epub 2003 Mar 17.

Sleat, D. E., Sohar, I., Gin, R. M., Lobel, P. Aminoglycoside-mediated suppression of nonsense mutations in late infantile neuronal ceroid lipofuscinosis. Europ. J. Paediatr. Neurol. 5 (Suppl A): 57-62, 2001.

Stephenson, J. Antibiotics show promise as therapy for genetic disorders. JAMA 285: 2067-2068, 2001.

Wilschanski, M., Famini, C., Blau, H., Rivlin, J., Augarten, A., Avital, A., Kerem, B., Kerem, E. A pilot study of the effect of gentamicin on nasal potential difference measurements in cystic fibrosis patients carrying stop mutations. Am. J. Respir. Crit. Care Med. 161: 860-865, 2000.

Wilschanski M, Yahav Y, Yaacov Y, Blau H, Bentur L, Rivlin J, Aviram M, Bdolah-Abram T, Bebok Z, Shushi L, Kerem B, Kerem E. Gentamicin-induced correction of CFTR function in patients with cystic fibrosis and CFTR stop mutations. N. Engl. J. Med. 349(15):1433-41, 2003.

Xi, B., Guan, F., Lawrence, D. S. Enhanced production of functional proteins from defective genes. J. Am. Chem. Soc. 126(18): 5660-1, 2004.

Yoshizawa, S., Fourmy, D., Puglisi, J. D. Structural origins of gentamicin antibiotic action. EMBO J. 17: 6437-6438, 1998.

U.S. Pat. No. 6,458,538 B1, Beckmann et al., Methods of assaying for compounds that inhibit premature translation termination and nonsense mediated RNA decay, issued Oct. 1, 2002.

What is claimed is:

1. A method for enhancing production in a subject of a functional protein from a gene disrupted by the presence of a premature stop codon in the coding region of the gene, comprising administering to the subject an amount of an agent effective to suppress the premature stop codon and an amount of an agent effective to increase transcription of the gene.

2. The method of claim 1, wherein the agent that suppresses the premature stop codon is an aminoglycoside antibiotic.

3. The method of claim 2, wherein the aminoglycoside antibiotic is selected from the group consisting of gentamicin, geneticin, paromomycin, hygromycin, G-418, kanamycin, amikacin and tobramycin.

4. The method of claim 3, wherein the aminoglycoside antibiotic is gentamicin.

5. The method of claim 3, wherein the aminoglycoside antibiotic is geneticin.

6. The method of claim 1, wherein the agent that increases transcription of the gene is an agent that activates a promoter of the gene.

7. The method of claim 6, wherein the agent that activates a promoter of the gene is a fluorinated quinolone.

8. The method of claim 7, wherein the fluorinated quinolone is ofloxacin.

9. The method of claim 6, wherein the agent that activates a promoter of the gene is thioguanine.

10. The method of claim 1, wherein the production of functional protein is enhanced by a factor of at least 7-fold relative to an untreated control.

11. The method of claim 10, wherein the production of functional protein is enhanced by a factor of at least 10-fold relative to an untreated control.

12. The method of claim 11, wherein the production of functional protein is enhanced by a factor of at least 20-fold relative to an untreated control.

13. The method of claim 12, wherein the production of functional protein is enhanced by a factor of at least 30-fold relative to an untreated control.

14. The method of claim 1, wherein the production of functional protein is enhanced by a factor of at least 2-fold relative to the production obtained using only the agent that suppresses the premature stop codon.

15. The method of claim 14, wherein the production of functional protein is enhanced by a factor of at least 3-fold relative to the production obtained using only the agent that suppresses the premature stop codon.

16. The method of claim 1, wherein the production of functional protein is enhanced to a level that corresponds to at least 10% of the level of functional protein generated from a corresponding native gene in which a premature stop codon is absent.

17. The method of claim 1, wherein the agent that suppresses the premature stop codon is administered at a dose lower than the dose that would be required to produce the same amount of functional protein in the absence of the agent that increases transcription.

18. The method of claim 17, wherein the lower dose of the agent that suppresses the premature stop codon results in decreased toxicity.

19. The method of claim 1, wherein the disruption of the gene is associated with a genetic disorder.

20. The method of claim 19, wherein the genetic disorder is selected from the group consisting of thalassemia, hemophilia A, hemophilia B, von Willebrand's disease, a p53 related cancer or disorder, a colorectal cancer, cystinosis, cystic fibrosis, Duchenne muscular dystrophy, Tay-Sachs disease, Wilms tumor, retinoblastoma, neurofibromatosis, ataxia telangiectasia, Hurler's syndrome, mucopolysaccharidosis I, and late infantile neuronal ceroid lipofuscinosis.

21. The method of claim 20, wherein the genetic disorder is ataxia telangiectasia.

22. The method of claim 21, wherein the agent that activates a promoter of the gene is a fluorinated quinolone.

23. The method of claim 22, wherein the fluorinated quinolone is ofloxacin.

24. The method of claim 21, wherein the agent that activates a promoter of the gene is thioguanine.

25. The method of claim 21, wherein the agent that suppresses the premature stop codon is gentamicin.

26. The method of claim 21, wherein the agent that suppresses the premature stop codon is geneticin.

27. The method of claim 19, wherein the genetic disorder is treated.

28. The method of claim 1, wherein the gene is a tumor suppressor gene.

29. The method of claim 28, wherein the tumor suppressor gene is BRCA1, BRCA2, PTEN, NF1, NF2, MLH1, MLH2, VHL, WT1, TSC1, TSC2, and/or ATM.

30. The method of claim 28, wherein the enhanced production of the functional protein is effective to treat a tumor in the subject.

31. A method for enhancing production in a subject of a functional protein, where production of the protein is disrupted by a genetic mutation, comprising administering to the subject an amount of an agent effective to suppress the genetic mutation and/or correct a defect caused by the mutation, and an amount of an agent effective to increase transcription of the gene.

32. The method of claim 31, wherein the genetic mutation is a missense, frame shift, and/or exon skipping mutation.

33. The method of claim 32, wherein the mutation is an exon skipping mutation.

34. The method of claim 33, wherein the agent that suppresses the exon skipping mutation and/or corrects a defect caused by the mutation is a synthetic hybrid peptide-nucleic acid molecule, a 2′-O-methyl phosphorothioate oligonucleotide combining an antisense sequence and an exonic splicing enhancer sequence, sodium butyrate, or aclarubicin.

35. The method of claim 31, wherein the agent that increases transcription of the gene is an agent that activates a promoter of the gene.

36. The method of claim 31, wherein the genetic mutation is associated with a genetic disorder.

37. The method of claim 33, wherein the mutation is associated with spinal muscular atrophy (SMA).

38. The method of claim 31, wherein the genetic disorder is treated.
